(19) United States
(12) Patent Application Publication
(10) Pub. No.: US 2024/0122412 A1
(43) Pub. Date: Apr. 18, 2024

(54) Apparatus for Shelling Eggs

(71) Applicant: Moba Group B.V., Barneveld (NL)

(72) Inventors: Leopoldo PESCHI, Treviso (IT); Fabio GUALTIERI, Milaan (IT); Paul BUISMAN, Scherpenzeel (NL)

(21) Appl. No.: 18/254,810

(22) PCT Filed: Nov. 26, 2021
(86) PCT No.: PCT/IB2021/060997

§ 371 Date: May 26, 2023

(30) Foreign Application Priority Data
Nov. 27, 2020 (IT) 102020000028796

(51) Int. Cl. A47J 43/14; A23J 1/09; A61L 2/10; A61L 2/26; B65G 45/10; B65G 47/44

(52) U.S. Cl. CPC A47J 43/145; A23J 1/09; A61L 2/10; A61L 2/26; B65G 45/10; B65G 47/44; A61L 2202/11; B65G 2201/0208; B65G 2207/26

(57) ABSTRACT

The present invention relates to an apparatus (10) for shelling eggs which includes: —a casing (11) defining a shelling compartment (A) and an unloading compartment (B); —a shelling device (12) housed in the shelling compartment (A) and configured to open egg shells; —an unloading device (13) housed in the unloading compartment (B) and comprising at least one hopper member (14a, 14b, 14c) suitable for receiving and unloading a shelling product consisting of albumen, yolk or a mixture of albumen and yolk; —a transport device (15) comprising conveyors (16, 17) configured to separately transport albumen and yolk from the shelling device (12) to the unloading device (13); —sanitizing devices (18, 19a, 19b, 20, 30) housed in the casing (11) and adapted to emit ultraviolet radiation and which comprise a first sanitizing device (18) positioned to irradiate operating surfaces of the at least one hopper member (14a, 14b, 14c), to reduce a bacterial load on said operating surfaces which are surfaces that come into contact with yolk and/or albumen during the operation of the apparatus (10).

### TECHNICAL FIELD

This invention relates to an apparatus for shelling eggs and especially for obtaining a mixed product, yolk and albumen, or which makes yolk and albumen available separately, where the shelling product allows a longer shelf life to be obtained, other treatment conditions being equal, compared to prior art solutions.

### BACKGROUND ART

In the field of treatment of food products, both the need to obtain a long shelf life and to obtain products with organoleptic properties close to those of the fresh product are strongly felt.

In the field of treatment of eggs, long shelf life is achieved by adjusting the pasteurization parameters.

In other words, the product to be pasteurized, be it a mixture of yolk and albumen or pure albumen, is made to touch a hot surface from which it receives the pasteurization heat.

The pasteurization temperature must be maintained, depending on the product to be pasteurized, in a range substantially between 57° C. and 72° C. to avoid degrading the product at higher temperatures or to avoid obtaining insufficient pasteurization by using lower temperatures.

The aim of pasteurization is to reduce the bacterial load present in the product to be pasteurized, therefore, together with the temperature, a key parameter of the pasteurization process is the duration of the isothermal period, that is, the duration of the product being kept at the pasteurization temperature.

Within the above-mentioned temperature limits, a longer isothermal period determines a greater reduction of the bacterial load, therefore, depending on the intended use of the final product, higher pasteurization temperatures are chosen combined with shorter isothermal periods or, vice versa, longer isothermal periods combined with lower temperatures.

According to the bacterial load contained in the product to be pasteurized, the pasteurization temperature and the relative isothermal period will be determined.

Higher temperatures with shorter isothermal periods benefit productivity, however lower pasteurization temperatures and longer isothermal periods favour the organoleptic qualities of the resulting product.

In general, the organoleptic qualities of the resulting product are better the less rigid the thermal pasteurization treatment is, that is, the lower the temperature and the shorter the isothermal period.

### SUMMARY OF THE INVENTION

The problem underlying the invention is therefore that of improving the shelf life of a product deriving from shelling of eggs, be it a mixture of egg albumen and yolk or only albumen or only yolk, obtaining organoleptic qualities of the product which are significantly comparable with those of the fresh product.

The task of an egg shelling apparatus according to the invention is therefore to solve this problem.

Within this aim, an object of the invention is to provide an apparatus for shelling eggs which can guarantee a bacterial load of the final product, with the same raw material, which is considerably lower than that obtainable by means of traditional plants and processes.

Within this task, an aim of the invention is to provide an apparatus for shelling eggs which, for the same quality of the final product, allows shorter production times to be obtained with respect to prior art plants and processes.

Another aim of the invention is to provide an apparatus for shelling eggs which is simple to implement and easy to use.

This aim, as well as these and other aims which will emerge more fully below, are attained by an apparatus according to the appended independent claim.

Detailed features of an apparatus for shelling eggs according to the invention are indicated in the dependent claims.

Further features and advantages of the invention will emerge more fully from the description of a preferred but not exclusive embodiment of an apparatus for shelling eggs, illustrated by way of non-limiting example in the accompanying drawings, in which:

### BRIEF DESCRIPTION OF THE DRAWINGS

FIG. 1 shows a diagram of an apparatus for treating eggs according to the invention;

FIG. 2 shows a detail of an apparatus for treating eggs according to the invention, relating to a piston filter, in a median section;

FIG. 3 illustrates a detail of an apparatus for treating eggs according to the invention, relating to a module for transporting yolk and albumen;

FIG. 4 shows a detail of an apparatus for treating eggs according to the invention, relating to a series of modules of FIG. 3;

FIG. 5 is a perspective view of a detail of an apparatus for treating eggs according to the invention, relating to a shelling device and a transport device, with some parts removed so as to better highlight others.

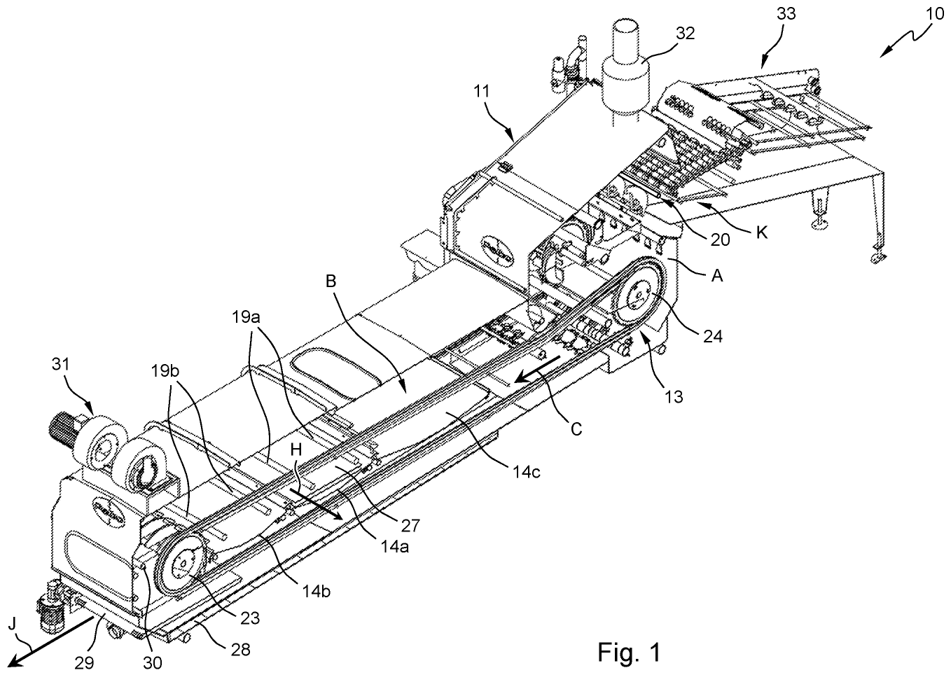

### DETAILED DESCRIPTION

With particular reference to the accompanying drawings, the reference number 10 denotes in its entirety an apparatus for shelling eggs which comprises:
a casing 11 defining a shelling compartment A and an unloading compartment B;
a shelling device 12 housed in the shelling compartment A and configured to open egg shells;
an unloading device 13 housed in the unloading compartment B and comprising at least one hopper member 14a, 14b, 14c suitable for receiving and unloading a shelling product consisting of albumen, yolk or a mixture of albumen and yolk;
a transport device 15 comprising conveyors 16, 17 configured to separately transport albumen and yolk from the shelling device 12 to the unloading device 13;
sanitizing devices 18, 19a, 19b, 20, 30 housed in the casing 11 and adapted to emit ultraviolet radiation and which comprise a first sanitizing device 18, and possibly an accessory sanitizing device 19b, positioned to irradiate operating surfaces of the hopper member 14a, 14b, respectively, to reduce a bacterial load on the operating surfaces of the latter, which consist of surfaces that come into contact with the yolk and/or albumen during the operation of the apparatus.

The sanitizing devices 18, 19a, 19b, 20, 30 can comprise one or more ultraviolet lamps which can be tubular.

For example, these ultraviolet radiations can have a power of between 100 and 2500 μW/cm² for a time between 10 and 60 seconds, to counteract bacterial proliferation and, moreover, each shelling device is sanitized, following each shelling, for example by spraying with a spray of ozonated water or with an aqueous solution of hydrogen peroxide, for example at 1.4%.

Thanks to the irradiation carried out by the sanitizing devices 18, 19a, 19b, 20, 30, in an apparatus 10 according to the invention it is possible to significantly reduce the bacterial load contained in the shelling product so that an egg treatment plant comprising an apparatus 10 allows, with the same other functional parameters, a product to be obtained with a lower final bacterial load and, therefore, with a longer shelf life.

Moreover, in an egg treatment plant comprising an apparatus 10 according to the invention, it is possible to adopt lower pasteurization temperatures and/or lower times at the pasteurization temperature, because the bacterial load to be reduced is lower.

Thus, the use of an apparatus according to the invention in a plant for treating eggs allows, with the same other functional parameters, to obtain a product with better organoleptic qualities than prior art plants.

The sanitizing devices 18, 19a, 19b, 20, 30 can comprise a third sanitizing device 20 positioned to irradiate working parts of the shelling device 12 where these working parts include parts able to come into contact with an egg or with the shell and/or with the shelled egg during operation of the apparatus 10, this eliminating or reducing any contamination of the shell and therefore of the shelling product, reducing its bacterial load.

The shelling device 12 can comprise at least one opening member 34, of a traditional type and configured to open an egg shell and cause the yolk and albumen to fall.

The conveyors 16, 17 may comprise:
at least a first conveyor 16 having a plurality of seats each adapted to house yolk;
at least a second conveyor 17 having a plurality of seats each suitable for housing albumen.

Each opening member 34 can be placed above the first conveyor 16 to discharge a yolk for each seat of the latter.

The first conveyor 16 can be placed above the second conveyor 17 to discharge albumen into the seats of the second conveyor 17.

The said operating surfaces of the conveyors 16, 17 can comprise said seats which, therefore, are irradiated by ultraviolet radiation during the operation of the apparatus 10, to reduce any bacterial load present on them.

The first sanitizing device 18 can be positioned in such a way as to irradiate said seats before they are engaged respectively by a yolk and by an albumen, during the operation of the apparatus 10.

In this way the possibility of contamination, or its extent, of the shelling product received by the shelling device 12 is limited to the full.

The first sanitizing device 18 can be placed side by side with the shelling device 12 and, in particular, it can be placed upstream of the latter with respect to a direction of advancement C of the seats of the conveyors 16 and 17, to sanitize the latter just before being filled.

The conveyors 16, 17 may comprise:
a plurality of modules 22, each comprising at least one of the seats, and which can be connected in sequence to form respectively a first chain member and a second chain member, which are closed in a ring;
a first transmission unit 23 and a second transmission unit 24 to which the chain members are connected and between which the latter extend, defining a forward section D and a return section E of the chain members which extend from the shelling compartment A to the unloading compartment B.

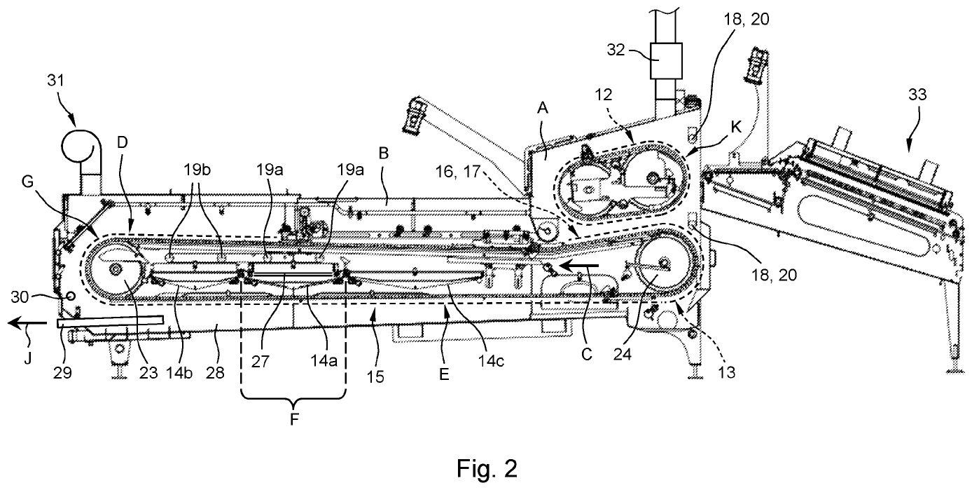

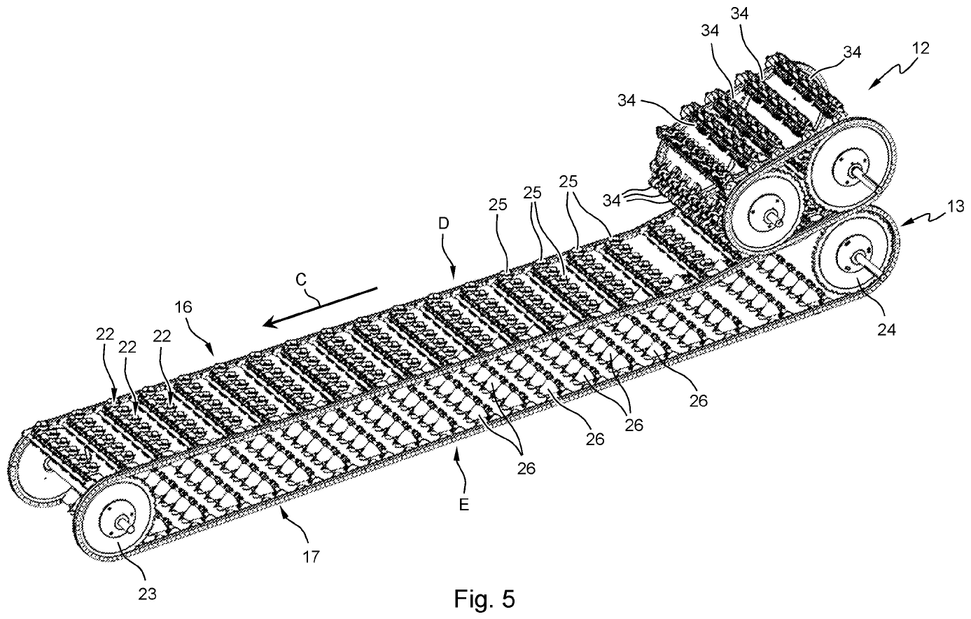

For example, FIG. 5 illustrates a shelling device 12 and an unloading device 13, which in FIG. 2 are schematized by a dashed line.

The unloading device 13 of FIG. 5 has some parts removed to facilitate their intelligibility.

In FIG. 5, in particular, the conveyors 16 and 17 are seen, where the first conveyor 16 is seen in the forward section D where it hides the second conveyor 17 which is under it.

On the contrary, in the return section E the second conveyor 17 is visible and hides the first conveyor 16 which is under it.

In the example of FIG. 5 it can be seen how the conveyors 16 and 17 are integrated in a single product which has a plurality of modules 22, which form lines and rows respectively transversal and longitudinal with respect to the direction of advancement C.

The first chain member and the second chain member can constitute a single product, that is, consist of a single chain member.

The ultraviolet lamps of the sanitizing devices 18, 19a, 19b, 20, 30 can extend transversely to the direction of advancement C to irradiate a plurality of modules 22 side by side which, during operation of the system, move along the direction of advancement C.

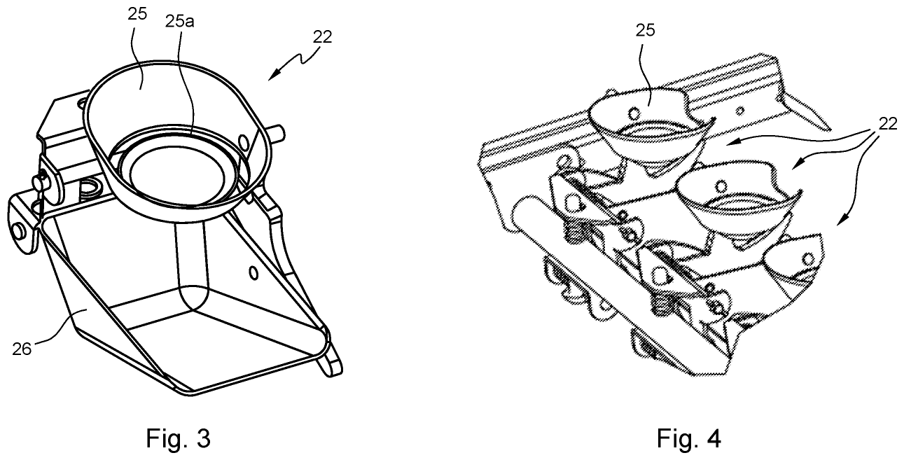

The seats of the conveyors 16, 17 can be defined respectively by a first spoon element 25 and a second spoon element 26, for example like those illustrated in FIGS. 3, 4 and 5.

Each module 22 can comprise a first spoon element 25 and a second spoon element 26 which can be reciprocally positioned in such a way that, along the forward section D, the first spoon element 25 is positioned above the second spoon element 26, when the apparatus 10 is in use.

The first spoon element 25 can be configured in such a way as to retain a yolk and discharge the albumen of an egg.

For example, it can have at least one opening or holes 25a positioned and configured not to allow the passage of a whole yolk but to allow the passage of the albumen that surrounds it.

The second spoon element 26 can be configured so as to receive an albumen unloaded from the first spoon element 25 of the same module 22.

The unloading device 13 can comprise:
an emptying member operable to interfere with the second conveyor 17 in a first unloading position F of the unloading compartment B;
a first hopper member 14a of the at least one hopper member 14a, 14b, 14c which is positioned so that, in use, it is under the second conveyor 17 in the first unloading position F to receive the contents of the seats of the second conveyor 17.

The emptying member can be configured to cause, following its actuation, a fall of the contents of the seats of the first conveyor 16 when these pass through the first unloading position F during the operation of the apparatus 10.

For example, the emptying member can be configured to rotate the second spoon 26 in such a way as to cause it to overturn to make its contents fall into the first hopper member.

The emptying member and the conveyors 16 and 17 can be of a traditional type and, therefore, not further described or illustrated.

The apparatus 10 may comprise a first filtering device 27 positioned in such a way that in use it is located above the first hopper member 14a to filter and retain eggshell fragments and allow the passage of the shelling product.

The second sanitizing device 19 can face the first filtering device 27 and/or the first hopper member 14a to reduce any bacterial load present on them.

The unloading device 13 can comprise a second hopper member 14b and/or a third hopper member 14c which can be housed in the unloading compartment B and positioned, with respect to the conveyors 16 and 17, so that when the apparatus is in use they are under conveyors 16 and 17.

The second sanitizing device 19b can face the second hopper member 14b to sanitize it.

The unloading device 13 can be configured to cause the falling of shelling product from the seats of both the conveyors 16 and 17 into the second hopper member 14b, to simultaneously collect both the yolk and the albumen together.

Moreover, the unloading device 13 can be configured to cause the falling of shelling product selectively from specific seats of the conveyors 16 and 17 into the third hopper member 14c, to collect waste yolks and/or albumen.

In this case, the selection of said specific seats can be carried out by an operator or by a selection device, possibly equipped with a viewing system facing said seats, which is integrated or connected with the unloading device 13 to operate it so as to unload their contents from said specific seats when such content is identified as to be discarded.

The unloading device 13 can comprise:
a tank 28 positioned in the unloading compartment B in such a way that in use it is under the first conveyor 16 to receive the shelling product from the latter;
a refrigeration device, not shown, thermally connected to the tank 28 to cool it to a temperature not higher than 15° C. and preferably between 0° C. and 4 C, for example by means of a cooling-jacket with recirculation of refrigerant associated with the tank 28.

The apparatus 10 can be configured to unload the shelling product from the first conveyor 16 into a second unloading position G of the unloading compartment B.

The apparatus 10 can comprise a second filtering device 29 positioned in such a way as to receive shelling product from the first conveyor 16 unloaded in the second unloading position G, to filter and retain eggshell fragments and allow the passage of the shelling product.

The apparatus 10 can comprise a fourth sanitizing device 30 of said sanitizing devices 18, 19a, 19b, 20, 30 which faces the second filtering device 29 to reduce any bacterial load present on it.

The first filtering device 27 and/or the second filtering device 29, respectively, can comprise a mesh or perforated conveyor belt which can be operated to drag, in an ejection direction H, J, fragments of eggshell filtered during the operation of the apparatus 10.

The ejection direction H of the first filtering device 27 can be transversal and preferably perpendicular to the direction of advancement C.

The ejection direction H of the second filtering device 29 can be parallel to the direction of advancement C where the second unloading position G is located at one end of the transport device 15 distal to the shelling device 12.

The apparatus 10 may comprise ventilation means which are in aeraulic communication with the interior of the casing 11 to force a flow of gas into the latter.

These ventilation means may comprise or consist of one or more fans 31.
said gas is optionally filtered air or a gaseous mixture containing ozone.

Said filtered air can preferably consist of a flow of air filtered by means of a HEPA filter which can be fixed to the fans 31 to be crossed by the flow of air forced by the latter.

The apparatus 10 can comprise a suction unit 32 which is in aeraulic connection with the interior of the casing 11 and is configured to suck a gas contained in the latter.

In particular, the apparatus 10 is preferably configured to operate the ventilation means and/or the suction unit 32 in such a way that the inside of the casing 11 is in depression with respect to the relative environment outside if the gas introduced by the ventilation means in the casing 11 includes ozone.

The apparatus 10 may also comprise a loading device 33 configured to load eggs into the shelling device 12 through an inlet compartment K of the casing 11.

The inlet compartment K can be provided at a first end of the casing 11 where at a second end of the same, distal from the first end with respect to the direction of advancement C, an ejection opening L can be provided through which the second filtering device 29 can eject waste material from the casing 11.

The ultraviolet lamps of the first sanitizing device 19a and of the second sanitizing device 19b are preferably placed above the sides of the first hopper member 14a and of the second hopper member 14b respectively, for example in such a way that these lamps are spaced along the direction of advancement C so as to leave a free space between them for the falling of shelling product into the first and second hopper members 14a, 14b.

It is therefore understood how an apparatus according to the invention achieves the intended aims and objectives by improving the shelf life of a product deriving from the shelling of eggs, be it a mixture of albumen and yolk or only albumen or only yolk, obtaining organoleptic qualities of the product which are significantly comparable with those of the fresh product.

Furthermore, an apparatus for shelling eggs according to the invention allows a bacterial load of the final product to be guaranteed, with the same raw material, which is considerably lower than that obtainable by means of prior art systems and processes. Furthermore, an apparatus according to the invention allows, with the same quality of the final product, shorter production times to be obtained with respect to prior art plants and processes.

The invention thus conceived is susceptible to numerous modifications and variations, all of which fall within the scope of protection of the attached claims.

Further, all details may be replaced by other technically equivalent elements.

Where the operating and technical features mentioned are followed by signs or reference numbers, the signs or reference numbers have been used only with the aim of increasing the intelligibility of the description and claims themselves and, consequently, they do not constitute in any way a limitation to the interpretation of each element identified, purely by way of example, by the signs or reference numerals.

1. An apparatus for shelling eggs which includes:
a casing defining a shelling compartment (A) and an unloading compartment (B);
a shelling device housed in said shelling compartment (A) and configured to open egg shells;
an unloading device housed in said unloading compartment (B) and comprising at least one hopper member suitable for receiving and unloading a shelling product consisting of albumen, yolk or a mixture of albumen and yolk;
a transport device comprising conveyors configured to separately transport albumen and yolk from said shelling device to said unloading device;
sanitizing devices housed in said casing adapted to emit ultraviolet radiation and which comprise a first sanitizing device positioned to irradiate operating surfaces of said at least one hopper organ, to reduce a bacterial load on said operating surfaces, wherein said operating surfaces are surfaces that come into contact with yolk and/or albumen during the operation of said apparatus-.

2. The apparatus according to claim 1 wherein said sanitizing devices comprise a third sanitizing device positioned to irradiate working parts of said shelling device wherein said working parts comprise parts able to come into contact with an egg during the operation of said apparatus.

3. The apparatus according to claim 1 wherein said shelling device comprises at least one opening member configured to open an egg shell and cause the yolk and albumen to fall; said conveyors comprise:
at least a first conveyor having a plurality of seats each adapted to house a yolk;
at least a second conveyor having a plurality of seats each suitable for housing albumen;
wherein each of said opening member is arranged above one of said at least one first conveyor to unload a yolk for each seat of said first conveyor;
wherein each of said at least one first conveyor is arranged on top of one of said at least one second conveyor to unload albumen into the seats of said second conveyor;
the operating surfaces of said conveyors comprise said seats; said first sanitizing device being positioned in such a way as to irradiate said seats before they are engaged respectively by a yolk and an albumen, during the operation of said apparatus.

4. The apparatus according to claim 3 wherein said first sanitizing device is arranged side by side to said shelling device and arranged upstream of the latter with respect to a direction of advancement (C) of said seats, to sanitize the latter just before being filled.

5. The apparatus according to claim 1 wherein said unloading device comprises:
an emptying member operable to interfere with said second conveyor in a first unloading position (F) of said unloading compartment (B); said emptying member being configured to cause, following its actuation, a fall of the contents of the seats of said first conveyor when they pass through said first unloading position (F) during the operation of said apparatus;
a first hopper member of said at least one hopper member which is positioned so that, in use, it is under said second conveyor in said first unloading position (F) to receive the contents of the seats of said second conveyor.

6. The apparatus according to claim 5 which comprises a first filtering device positioned so that, in use, it is above said first hopper member to filter and retain eggshell fragments and allow the passage of said shelling product.

7. The apparatus according to claim 6 wherein a second sanitizing device of said sanitizing devices faces said first filtering device and said first hopper member to reduce any bacterial load present on them.

8. The apparatus according to claim 1 wherein said unloading device comprises:
a tank arranged in said unloading compartment in such a way that in use it is under said first conveyor to receive said shelling product from the latter;
a refrigeration device thermally connected to said tank to cool it to a temperature not higher than 15° C. and preferably between 0° C. and 4° C., for example by means of a cooling-jacket with recirculation of refrigerant associated with the tank.

9. The apparatus according to claim 8 configured for unloading said shelling product from said first conveyor into a second unloading position (G) of said unloading compartment (B); said apparatus comprising a second filtering device arranged in such a way as to receive shelling product from said first conveyor unloaded into said second unloading position (G), to filter and retain eggshell fragments and allowing the passage of said shelling product.

10. The apparatus according to claim 9 which comprises a fourth sanitizing device of said sanitizing devices which faces said second filtering device (29) to reduce any bacterial load present on it.

11. The apparatus according to claim 6 wherein said first filtering device and/or said second filtering device, respectively, comprises a mesh or perforated conveyor belt operable to drag, in an ejection direction (H, J), fragments of eggshell filtered during the operation of said apparatus.

12. The apparatus according to claim 1 which comprises ventilation means which are in aeraulic communication with the interior of said casing to force a gas to flow into the latter, wherein said gas is optionally filtered air or a gaseous mixture containing ozone; said filtered air consisting of a flow of air filtered by a HEPA filter.

13. The apparatus according to claim 1 comprising a suction unit which is in aeraulic connection with the interior of said casing and is configured to suck a gas contained in the latter.
